(19) United States
(12) Patent Application Publication
(10) Pub. No.: US 2013/0331740 A1
(43) Pub. Date: Dec. 12, 2013

(54) ULTRASONIC HEAD TRAP

(71) Applicant: Access Business Group International LLC, Ada, MI (US)

(72) Inventors: Bradley J. Pippel, Grandville, MI (US); Richard B. Bylsma, Ada, MI (US)

(21) Appl. No.: 13/909,518

(22) Filed: Jun. 4, 2013

Related U.S. Application Data
(60) Provisional application No. 61/657,316, filed on Jun. 8, 2012.

(51) Int. Cl. A61N 7/02

(52) U.S. Cl. CPC A61N 7/02
USPC 601/3; 601/2

(57) ABSTRACT

A wand for delivering therapeutic ultrasonic energy to a target area includes a module and a transducer. The transducer converts electrical energy into ultrasonic vibrations that are delivered to the module. The module includes a fluid chamber that houses an ultrasonic fluid. In order to reduce or eliminate the deleterious effects of bubbles that may form in the ultrasonic fluid, one or more bubble traps are defined in the fluid chamber that capture, store, and/or assist in keeping the bubbles outside of the direct transmission path of the ultrasonic energy through the fluid. One or more barriers may be included in the trap(s) that obstruct movement of the bubbles into the transmission path when the wand is tilted to different orientations. After passing through the fluid, the ultrasonic energy is delivered to the skin of a patient, or other surface to which the ultrasonic energy is being directed.

### CROSS-REFERENCE TO RELATED APPLICATIONS

This application claims priority to U.S. provisional patent application Ser. No. 61/657,316 filed Jun. 8, 2012 by Bradley J. Pippel and entitled Ultrasonic Head Trap, the complete disclosure of which is hereby incorporated herein by reference.

### BACKGROUND OF THE INVENTION

The present invention relates to ultrasonic devices, and more particularly to ultrasonic devices adapted to deliver heat energy to one or more targeted locations.

Ultrasonic energy may be used to deliver heat energy to a target by focusing the ultrasonic vibrations such that they arrive at the target in a concentrated manner. Such techniques may be useful for delivering ultrasound treatments to human or other animal tissue, particularly treatments intended to treat fatty tissues underlying the skin for cosmetic reasons. Systems for delivering ultrasonic energy to mammalian tissue in order to heat the tissue typically include an ultrasonic transducer, a control system for controlling the ultrasonic transducer, and a fluid chamber coupled to the ultrasonic transducer. The fluid chamber is adapted to focus the ultrasonic energy created by the transducer so that the energy is delivered to the mammalian tissue in the right location and with the desired level of concentration. The fluid inside of the fluid chamber may be in contact with a driving member of the ultrasonic transducer so that the ultrasonic energy is transmitted from the driving member into the fluid, which then passes the ultrasonic energy onto a contact membrane, or other structure that is in physical contact with the human or other mammal.

In the past, the transmission of ultrasonic energy through the fluid contained within the fluid chamber has been degraded by the presence of any air bubbles, or other gas bubbles. Such bubbles tend to interfere with the transmission of ultrasonic energy from the transducer to the target, decreasing the efficiency of the transfer and/or changing the focusing of the ultrasonic energy. In some cases, the interference of the bubbles may be so detrimental that the ultrasonic energy delivery system has to be replaced, leading to waste and increased costs.

### SUMMARY OF THE INVENTION

Accordingly, the present invention overcomes and/or reduces the disadvantages of bubbles associated with ultrasonic heat energy delivery systems. According to one aspect, one or more bubble traps are provided that capture, maintain, and/or assist in keeping bubbles in one or more locations outside of the direct ultrasonic energy transmission path through the fluid, thereby reducing or eliminating any interference the bubbles would otherwise create with the transmission of ultrasonic energy through the fluid. In other aspects, an ultrasonic energy delivering wand is provided that includes a bubble trap for storing bubbles out of the direct transmission path of the ultrasonic energy, and/or for capturing bubbles that may temporarily be in the direct transmission path. In yet other embodiments, a method may be provided for delivering ultrasonic energy in a manner that reduces interference from any bubbles present in an ultrasonic fluid medium through which the ultrasonic energy passes. The methods, apparatus, and systems of the present invention can be applied to any energy transmission media, whether fluid or otherwise, in which bubbles may form.

According to a first embodiment, an ultrasonic module for transmitting ultrasonic energy from a transducer to a target is provided. The module includes a first end for receiving ultrasonic energy from an ultrasonic transducer, and a second end for delivering ultrasonic energy to a target, a fluid chamber, and a bubble trap. The fluid chamber is positioned between the first and second ends and is adapted to contain an ultrasonic fluid. The bubble trap is adapted to hold bubbles formed in the ultrasonic fluid in a location outside of a direct ultrasonic energy transmission path defined between the first and the second ends.

The module may further include a seat defined at the first end for positioning an ultrasonically vibrating member, and a barrier surrounding the seat that obstructs bubbles in the bubble trap from escaping out of the bubble trap and into the transmission path. The barrier may prevent bubbles in the bubble trap from escaping out of the bubble trap and into the transmission path when the module is tipped from a vertical orientation to a tipped orientation, wherein the vertical orientation is defined by the first and second ends being vertically aligned, and the tipped orientation is defined by the first and second ends not being vertically aligned. In some embodiments, the tipped orientation may include angular deviations as great as ninety degrees from the vertical orientation such that bubbles are retained in the trap for all angular deviations up to at least ninety degrees from the vertical orientation.

The module may be configured to capture bubbles positioned in the transmission path whenever the module is tipped such that the first and second ends are not vertically aligned. The bubble trap may be positioned closer to the first end than to the second end. The fluid chamber may include a first section and a second section, wherein the first section includes a first inner dimension and the second section includes a second inner dimension that is smaller than the first inner dimension. The bubble trap may be defined in the first section. A barrier may be defined adjacent a junction of the first and second sections wherein the barrier obstructs bubbles from escaping out of the bubble trap and into the transmission path.

In some embodiments, both a first barrier and a second barrier may be provided. The first barrier may be defined near the first end and adapted to obstruct bubbles in the bubble trap from escaping out of the bubble trap and into the transmission path, and the second barrier may be defined between the first barrier and the second end, wherein the second barrier is also adapted to obstruct bubbles in the bubble trap from escaping out of the bubble trap and into the transmission path. The first barrier may be adapted to obstruct bubbles when the first end is positioned higher than the second end, and the second barrier may be adapted to obstruct bubbles when the second end is positioned higher than the first end.

The module may include a releasable fastening system for releasably securing the module to an ultrasonic transducer such that the module may be discarded without discarding the ultrasonic transducer. The module may also be attached to the ultrasonic transducer by way of a plurality of apertures adapted to receive a plurality of fasteners having a length sufficient to extend into a plurality of corresponding apertures defined in the ultrasonic transducer such that, when the fasteners are inserted into the apertures and the corresponding apertures, the module is secured to the ultrasonic transducer.

According to another embodiment, a hand-held ultrasonic wand for delivering heat energy to human tissue using ultrasonic energy is provided. The wand includes an ultrasonic transducer, a fluid chamber, and a bubble trap. The ultrasonic transducer is adapted to convert electrical energy into ultrasonic energy. The fluid chamber is coupled to the ultrasonic transducer and adapted to contain an ultrasonic fluid through which the ultrasonic energy is transmitted. The bubble trap is defined within the fluid chamber and is adapted to hold bubbles formed in the ultrasonic fluid in a location outside of a direct ultrasonic energy transmission path defined between the ultrasonic transducer and the first and second ends.

The wand may include a first barrier defined in the fluid chamber near the ultrasonic transducer that is adapted to obstruct bubbles in the bubble trap from escaping out of the bubble trap and into the transmission path, as well as a second barrier defined in the fluid chamber and spaced away from the second barrier wherein the second barrier also is adapted to obstruct bubbles in the bubble trap from escaping out of the bubble trap and into the transmission path. The first barrier may be adapted to obstruct bubbles when the wand is positioned above the human tissue, and the second barrier may be adapted to obstruct bubbles when the wand is positioned below the human tissue.

The wand may also include a releasable fastening system for releasably securing the fluid chamber to the ultrasonic transducer such that the fluid chamber may be discarded without discarding the ultrasonic transducer. A seat may be defined in the fluid chamber for receiving a driver of the ultrasonic transducer, and a contact membrane may be positioned at an end of the fluid driver in a location where it is adapted to contact human skin when delivering ultrasonic energy to the human tissue. The bubble trap may hold bubbles therein when the wand is tilted up to at least as much as ninety degrees from a vertical orientation, wherein the vertical orientation is defined by vertical alignment of the seat with the contact membrane. The wand may further be adapted to hold bubbles therein when the wand is rotated three-hundred and sixty degrees around a vertical axis while being tilted up to at least as much as ninety degrees from the vertical axis.

The wand may also be adapted to capture bubbles positioned in the transmission path whenever the wand is tipped such that the seat and the contact membrane are not vertically aligned. The fluid chamber may include a first section and a second section, wherein the first section is positioned adjacent the ultrasonic transducer, and the second section is positioned away from the ultrasonic transducer. The bubble trap may be defined in the first section. The first section may include a first inner dimension and the second section may include a second inner dimension that is smaller than the first inner dimension. The ultrasonic transducer may include a driver portion having a non-planar surface in contact with the ultrasonic fluid, wherein the non-planar surface urges the bubbles toward the bubble trap whenever the bubbles push against the non-planar surface of the driver portion.

According to yet another embodiment, a method for delivering ultrasonic energy to a target in order to create heat at the target is provided. The method includes generating ultrasonic vibrations in a driver portion of an ultrasonic transducer; transmitting the ultrasonic vibrations of the driver portion through a transmission path of an ultrasonic fluid that is in contact with the driver portion; and capturing bubbles in a location outside of the transmission path such that the bubbles do not interfere with the transmission of the ultrasonic vibrations through the fluid toward the target.

The method may further include retaining bubbles in the location outside of the transmission path when the ultrasonic transducer is tilted as much as at least ninety degrees from a vertical orientation. In another embodiment, the method may also include retaining bubbles in the location outside of the transmission path when the ultrasonic transducer is tilted as much as at least ninety degrees from a first vertical orientation, wherein the first vertical orientation is defined with the transducer positioned above the fluid; and also retaining bubbles in the location outside of the transmission path when the ultrasonic transducer is tilted as much as at least ninety degrees from a second vertical orientation, wherein the second vertical orientation is defined with the fluid being positioned above the transducer.

The method may also include storing the fluid in a fluid chamber that may be released from the transducer to allow a different fluid chamber to be attached to the transducer. The method may alternatively include storing the fluid in a fluid chamber that is permanently affixed to the transducer.

In any of the foregoing embodiments of the module, wand, and/or method, the target may be human tissue and the fluid and/or fluid chamber may be adapted to focus the ultrasonic energy in order to generate heat within the human tissue. A contact membrane may also be added to the fluid chamber that is adapted to contact the patient's skin when delivering ultrasonic energy to the tissue. The ultrasonic fluid may be an acoustic gel.

Before the various embodiments of the invention are explained in detail below, it is to be understood that the invention is not limited to the details of operation or to the details of construction and the arrangement of the components set forth in the following description or illustrated in the drawings. The invention may be implemented in various other embodiments and is capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a diagram of an ultrasonic energy delivery system according to one aspect of the invention that is adapted to deliver ultrasonic energy to a patient;

FIG. 2 is a diagram of the ultrasonic energy delivery system of FIG. 1 shown in an upside-down orientation in comparison to the orientation of FIG. 1;

FIG. 3 is a bottom view of an ultrasonic module that is part of the ultrasonic energy delivery system of FIGS. 1 and 2;

FIG. 4 is a sectional view of the ultrasonic module taken along the line IV-IV in FIG. 3;

FIG. 5 is a sectional view of the ultrasonic module taken along the line V-V in FIG. 3;

FIG. 6 is a sectional view of the ultrasonic module taken along the line VI-VI in FIG. 3;

FIG. 7 is the same sectional view as FIG. 4 but flipped upside down in order to illustrate a different potential orientation of the ultrasonic module;

FIG. 8 is a sectional view similar to FIG. 4 of a first alternative embodiment of an ultrasonic module according to another aspect of the present invention;

FIG. 9 is an elevational view of the ultrasonic module of FIG. 8 viewed from the direction of arrow 100 in FIG. 8;

FIG. 10 is a sectional view similar to FIG. 8 of a second alternative embodiment of an ultrasonic module according to another aspect of the present invention; and

FIG. 11 is an elevational view of the ultrasonic module of FIG. 10 viewed from the direction of arrow 100 in FIG. 10.

### DETAILED DESCRIPTION OF THE EMBODIMENTS

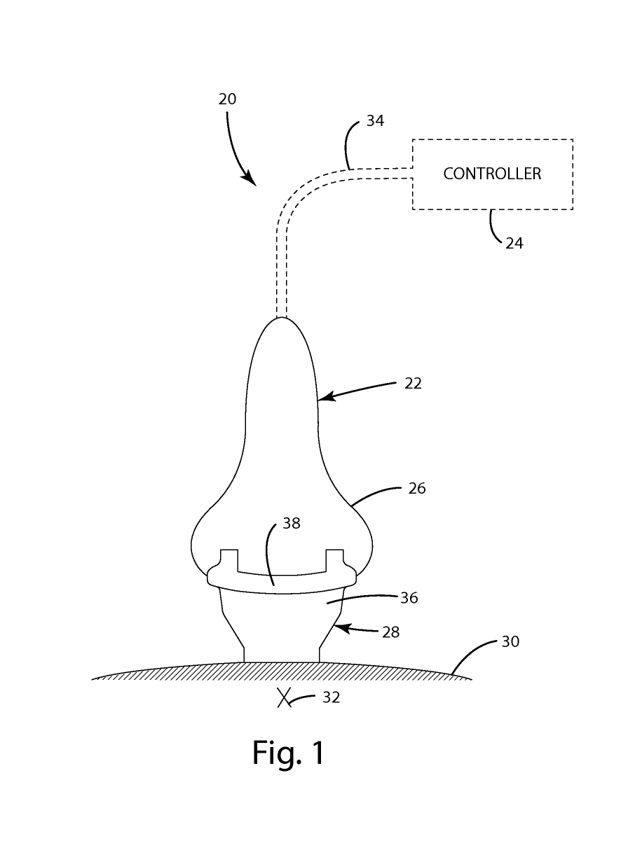

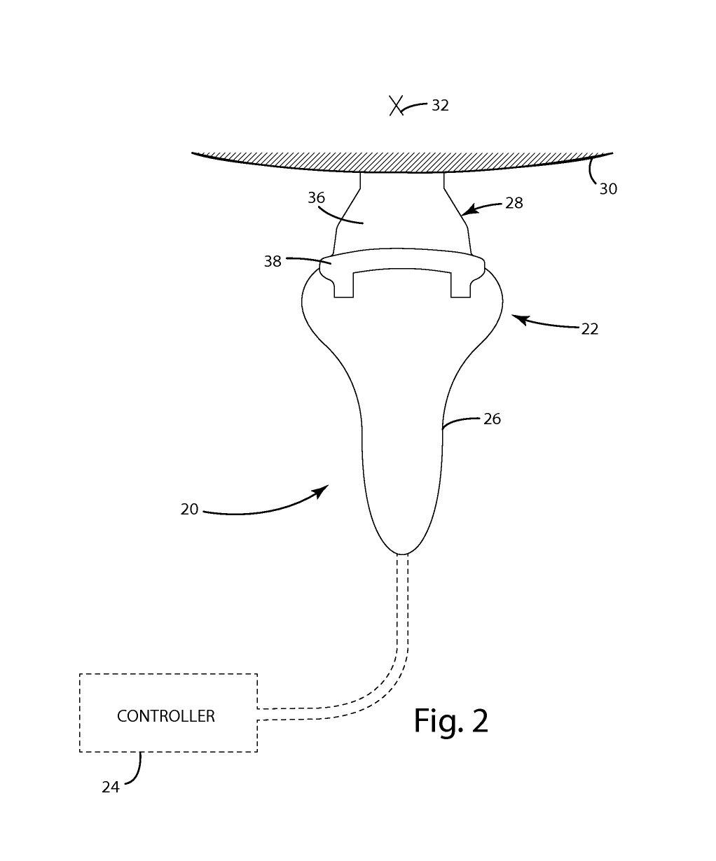

An ultrasonic delivery system 20 according to one embodiment is shown in FIG. 1. Ultrasonic delivery system 20 includes an ultrasonic wand 22 and an external controller 24. Wand 22 includes an ultrasonic transducer 26 and an ultrasonic module 28. Wand 22 is adapted to be applied to the skin 30 of a patient in order to deliver ultrasonic energy to a target area 32. The delivery of the ultrasonic energy to the target area 32 is adapted to generate heat, and, in some embodiments, the delivery of heat is useful for cosmetic purposes. It will, however, be understood by those skilled in the art that the delivery of ultrasonic energy to target area 32 may be for any useful purpose, whether cosmetic or otherwise. It will further be understood by those skilled in the art that the position of the target area 32 may vary from that shown in FIGS. 1 and 2. That is, in some embodiments, target area 32 may be positioned internally under the skin 30, such as shown in FIGS. 1 and 2, while in other embodiments target area 32 may be on or nearer to the surface of the patient's skin 30.

Controller 24 includes the electrical circuitry necessary for controlling wand 22 and delivering ultrasonic energy to target area 32. In some embodiments, controller 24 may be external to wand 22, such as is shown in FIGS. 1 and 2. However, in other embodiments, controller 24 may be incorporated inside of wand 22. When so incorporated, it may be possible to dispense with control cable 34. Indeed, in such an embodiment, wand 22 could be completely cable free (if an internal source of power is provided), or it may include only a power cable. The circuitry of controller 24, whether internal or external, could be any conventional circuitry suitable for implementing the desired delivery of ultrasonic energy to a target area.

Transducer 26 is adapted to convert electrical energy into ultrasonic vibrations that may be focused and delivered to the target area 32. Transducer 26 may utilize any known or conventional techniques for converting electrical energy to ultrasonic energy. In one embodiment, transducer 26 may include piezoelectric material that physically changes shape when an electrical voltage is applied thereto. By applying an appropriately varying voltage to the piezoelectric material, the material may be driven to vibrate with ultrasonic frequency. The ultrasonic vibrations created by transducer 26 are delivered to module 28, which focuses the ultrasonic energy and delivers it to the target area 32.

Wand 22 is adapted to be used in any desired orientation. That is, wand 22 may direct ultrasonic energy to target area 32 when wand 22 is positioned above the target area 32 (as shown in FIG. 1), or when wand 22 is positioned underneath the target area 32 (as shown in FIG. 2), or in any of the intermediate orientations (not shown) defined between the two vertical orientations shown in FIGS. 1 and 2. Thus, wand 22 can deliver ultrasonic energy to a target area no matter what its orientation is with respect to the target area.

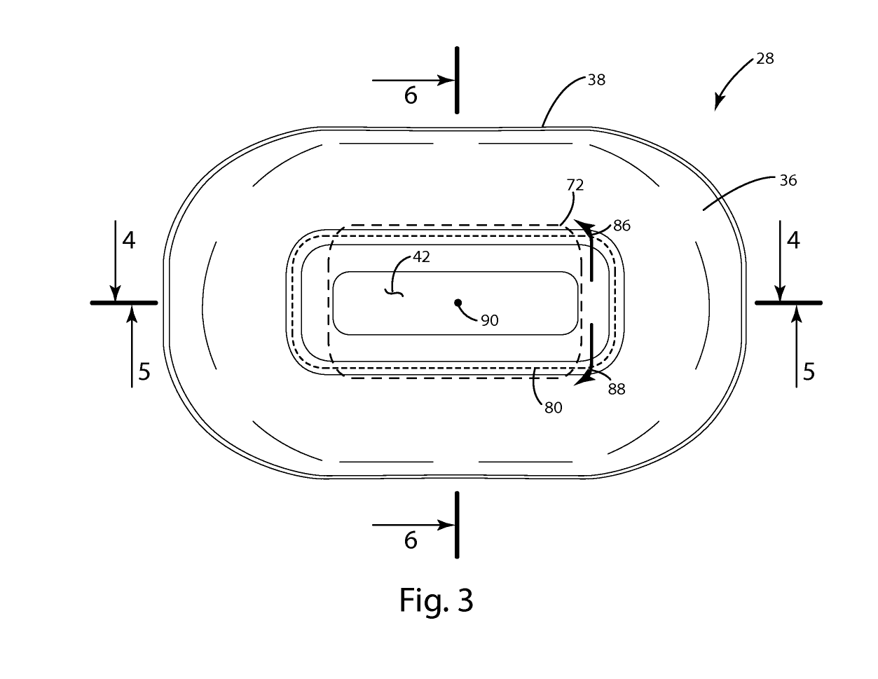

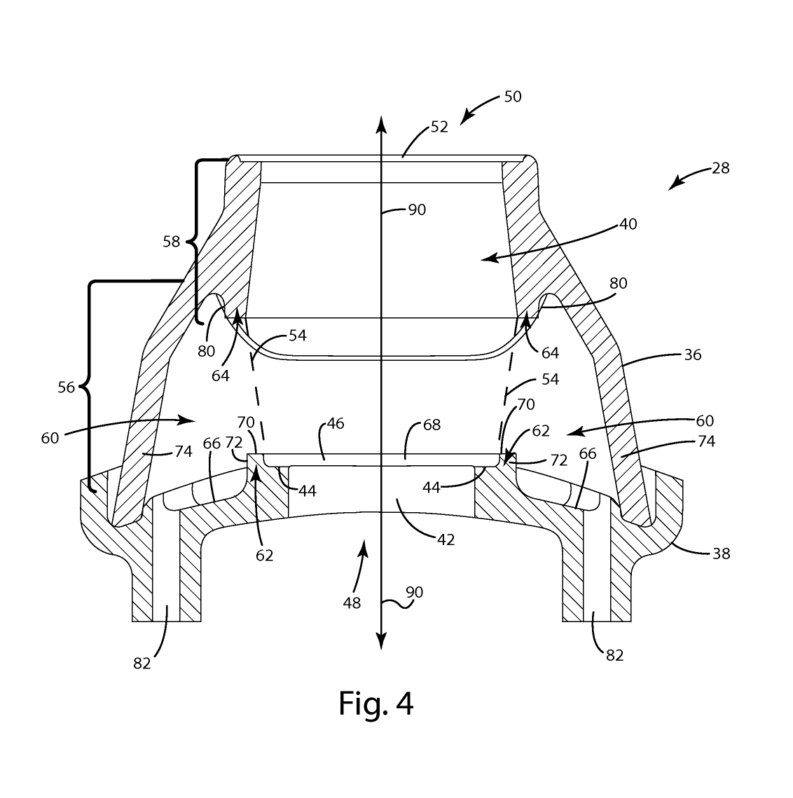

FIGS. 3-7 illustrate module 28 in greater detail. Module 28 includes a receiving end 48 and a delivery end 50 (FIGS. 4-7). Receiving end 48 is adapted to receive ultrasonic vibrations from transducer 26, while delivery end 50 is adapted to deliver the ultrasonic vibrations to the patient and, more specifically, target area 32 of the patient. Module 28 includes a base 36 positioned at delivery end 50 and a cover 38 that is positioned at receiving end 48. Cover 38 is adapted to be secured to base 36 such that the cover 38 and base 36 may be secured together to thereby define a fluid chamber 40 when so secured (FIGS. 4-7). A transducer aperture 42 is defined in cover 38 and dimensioned to receive a head portion of transducer 26. Cover 38 further defines a seat 44 that is dimensioned to receive a driver or driver portion 46 of transducer 26.

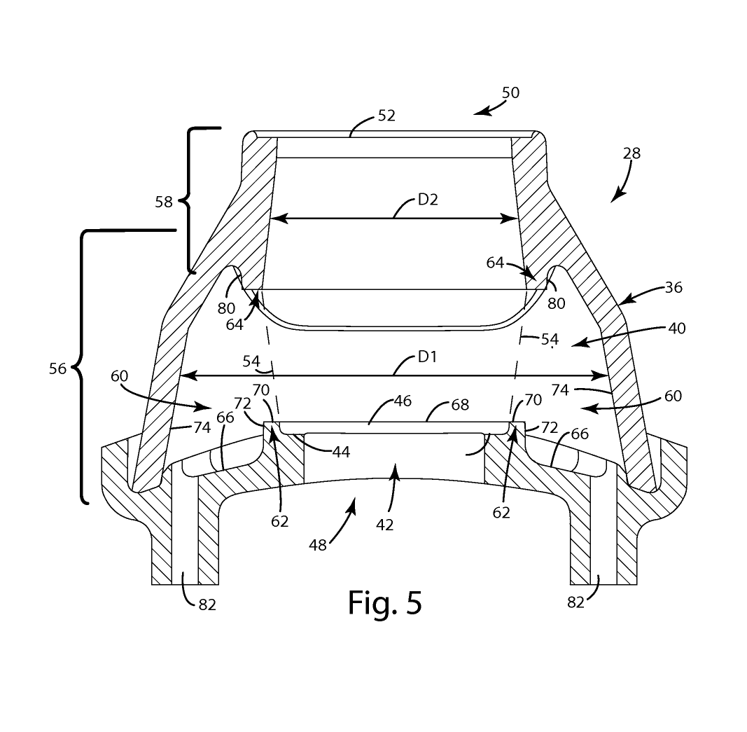

Fluid chamber 40 includes a first section 56 and a second section 58 (FIG. 4). First section 56 is positioned adjacent receiving end 48 of module 28 and abuts second section 58 on a side opposite receiving end 48. Second section 58 is defined adjacent delivery end 50 of module 28 and abuts first section 56 on an opposite side. As will be discussed in greater detail below, a bubble trap 60 is defined around the interior perimeter of first section 56 that helps to prevent bubbles formed in the ultrasonic fluid inside of fluid chamber 40 from migrating into a transmission path 54 of the ultrasonic energy. First section 56 includes a first interior dimension D1 while second section 58 includes a second interior dimension D2 (FIG. 5). While the precise value of interior dimensions D1 and D2 will vary depending upon the height at which they are measured (i.e. how far toward or away from receiving end 48 and/or delivery end 50), it can be seen that dimension D1 is larger than dimension D2 at all of the different heights.

Driver 46 may be made of piezoelectric material such that an appropriately varying voltage applied thereto causes it to vibrate at an ultrasonic frequency, or it may be made of another material that is caused to ultrasonically vibrate due to its physical contact with another vibrating structure within transducer 26. Regardless of the construction of transducer 26, when wand 22 is activated, the head portion of the transducer will cause driver 46 to vibrate at an ultrasonic frequency.

The vibration of driver portion 46 causes a corresponding vibration of an ultrasonic fluid that is contained within fluid chamber 40. The ultrasonic fluid may be any known or conventional fluid used for transmitting ultrasonic vibrations. The ultrasonic fluid may include fluids with high viscosity, as well as fluids with low viscosity, and also fluids exhibiting viscoelastic properties. In one embodiment, the fluid may be an ultrasonic gel. Some examples of fluids that may be used include water or glycerin, although it will be understood by those skilled in the art that other fluids may be used, and that this list of acceptable fluids is only a non-limiting list of examples. The term “fluid” and/or “ultrasonic fluid” as used herein will refer to not only liquids (which may have any range of viscosity), but also to any transmission media in which bubbles may form, including, but not limited to, colloids, emulsions, substances exhibiting mesophases, and other states of matter.

The ultrasonic vibrations created in the ultrasonic fluid travel away from driver portion 46, which is positioned at receiving end 48 of module 28, toward a contact membrane 52 that may be positioned at the extreme end of delivery end 50. Contact membrane 52 may be any conventional material that is adapted to contact the patient's skin and to transfer the ultrasonic vibrations to the target area within the patient. Examples of some suitable materials that may be used for contact membrane 52 include polycarbonate, polyetherimide or polypropylene, although it will be understood by those skilled in the art that other materials may also be used.

The transmission of ultrasonic energy from driver portion 46 to contact membrane 52 follows a direct transmission path 54 that is highlighted in FIG. 4. More specifically, the outer boundaries of transmission path 54 are shown in FIG. 4 in dashed lines. These outer boundaries are generally defined by straight lines that extend from the outer edges of driver portion 46 to the outermost interior edges of second section 58.

The ultrasonic waves created in the ultrasonic fluid initially pass through an interior region of first section 56 before continuing on through second section 58. While it is expected that the ultrasonic vibrations created by driver portion 46 will also travel outside of transmission path 54, transmission path 54 defines the direct transmission path between driver portion 46 and contact membrane 52. It is within this direct transmission path 54 that any bubbles are desirably removed. Bubbles positioned outside of this transmission path 54 will have minimal, if any, effect on the transmission of ultrasonic energy to target area 32.

As was noted, bubble trap 60 is defined generally in first section 56 of base 36. More specifically, bubble trap 60 is defined around the outside perimeter of transmission path 54 such that bubbles inside of bubble trap 60 do not cause any significant interference with the flow of ultrasonic energy down transmission path 54. While the precise shape of bubble trap 60 may vary from one embodiment to another, in the examples shown in FIGS. 4-6, bubble trap 60 includes a first barrier 62 positioned around the edges of seat 44 and a second barrier 64 positioned at the junction of first section 56 with second section 58. Both first and second barriers 62 and 64 extend around the entire interior perimeter of fluid chamber 40. In other words, first barrier 62 completely circumscribes seat 44, while second barrier 64 extends completely around the fluid chamber 40 such that it forms a single continuous ridge-like structure.

First and second barriers 62 and 64 serve to obstruct bubbles contained within trap 60 from migrating into transmission path 54. This obstruction is carried out by a combination of gravity and the shape of first and second barriers 62 and 64. In some situations, it is expected that wand 22 will be oriented such that transducer 26 will be positioned either wholly or partially above module 28 (such as shown in FIG. 1). In other situations, wand 22 may be held in an orientation in which module 28 is positioned above transducer 26, such as that illustrated in FIG. 2. Wand 22 may also be held in an intermediate orientation between those illustrated in FIGS. 1 and 2. The operation of bubble trap 60 will be explained more fully below with respect to both of the orientations shown in FIGS. 1 and 2, as well as the intermediate orientations.

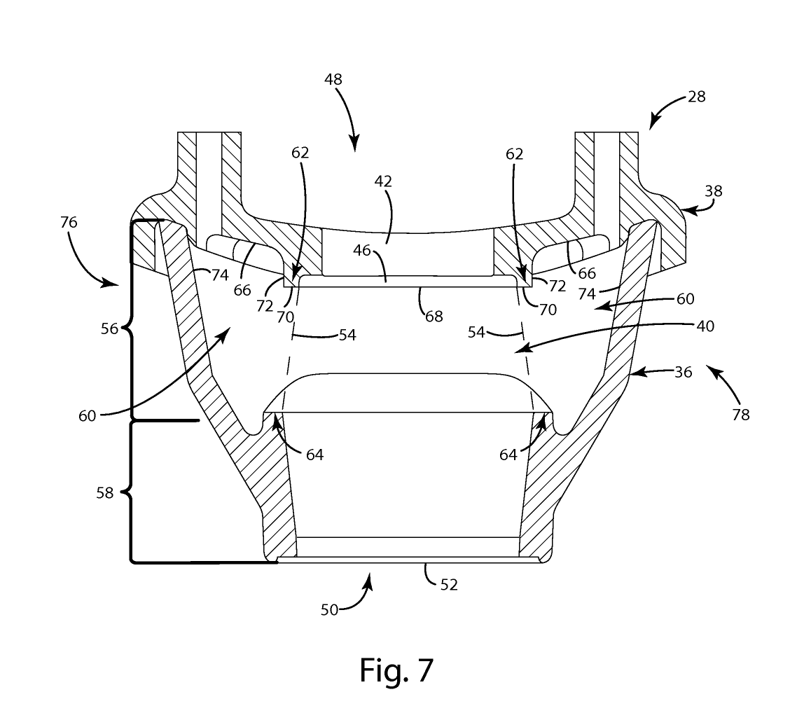

Beginning with the orientation shown in FIG. 1, in which module 28 is oriented in the same manner as in FIG. 7, the operation of bubble trap 60 will be explained. While in this orientation, or any intermediate orientation in which transducer 26 is positioned above module 28, any bubbles formed in the ultrasonic fluid will move upward (opposite the direction of gravity) toward cover 38. Depending upon the initial position of the bubble, it will continue to move upward until it impacts one of a top surface 66 of bubble trap 60, an exterior edge 68 of driver portion 46, or a flat surface 70 of first barrier 62 (see FIG. 7). If the bubble impacts top surface 66 of trap 60, the shape of trap 60—including first barrier 62—will tend to retain the bubble therein such that it cannot escape and migrate into transmission path 54. This will be explained in greater detail below.

If the bubble impacts either exterior edge 68 of driver portion 46 or flat surface 70 of first barrier 62, the bubble may tend to stop and remain in that position provided two conditions are met. First, the wand 22 cannot be tilted, and second, there can be no fluid movement within fluid chamber 40. Because both of these conditions are unlikely to occur for any extended period of time (due to a person moving and/or tilting wand 22 during use), any bubbles positioned adjacent exterior edge 68 of driver portion 46 or flat surface 70 of first barrier 62 will eventually migrate in one direction or another toward bubble trap 60. Once the bubble has migrated past barrier 62, it will become trapped in trap 60. More specifically, once the bubble moves past barrier 62, the tendency of the bubble to move upwardly (i.e. opposite the force of gravity) will cause the bubble to move into contact with top surface 66 of trap 60.

Once a bubble is in the top portion of trap 60 (i.e. near top surface 66), it will be prevented from migrating out of the trap by either a side wall 72 of barrier 62 or an outer wall 74 of trap 60 (FIG. 7). Which of these two walls 72 and 74 initially prevents the bubble from escaping from trap 60 will depend upon how wand 22, and thus module 28, is tipped, as well as the initial position of the bubble. Suppose, for example, that module 28—as shown in FIG. 7—is tipped such that a left side 76 is raised higher than a right side 78, and also suppose that a bubble is initially positioned in the portion of trap 60 adjacent right side 78. This bubble will initially migrate toward side wall 72 of barrier 62. Once it impacts side wall 72, however, it will be prevented from migrating further toward transmission path 54 by side wall 72. In fact, side wall 72 will continue to obstruct any movement of the bubble toward transmission path for any tipping of module 28 that is less than ninety degrees.

When module 28 is rotated ninety degrees (clockwise with respect to FIG. 7), side wall 72 will no longer be vertical as shown in FIG. 7, but will instead be horizontal. The upward vertical force of the bubble will therefore still urge the bubble toward side wall 72, however, no physical structure will prevent the bubble from moving laterally. Further, if module 28 is tipped more than ninety degrees, side wall 72 will be angled such that it will urge a bubble pressing against it toward transmission path 54. However, any such bubble that moves beyond side wall 72 toward transmission path will likely move completely through transmission path 54 all the way over to the portion of bubble trap 60 positioned near left side 76 because this portion of trap 60 will be positioned higher than any fluid in transmission path 54, and the bubble's natural tendency to move to the highest position in the fluid will therefore carry it into this portion of trap 60.

Thus, if module 28 is tipped or rotated in a clockwise fashion (with respect to FIG. 7) any amount equal to or less than ninety degrees, any bubble that initially starts out on a right side 78 of trap 60 will be prevented from escaping from trap 60 by barrier. Further, if module 28 is rotated clockwise more than ninety degrees, such a bubble may escape out of trap 60, but such escape will likely only be temporary as the bubble floats completely through transmission path 54 and eventually re-enters trap 60 on an opposite side.

It should be further pointed out that, even in some situations where wand 22 is tipped more than ninety degrees in a clockwise fashion (with reference to FIG. 7), a bubble positioned initially in the right side 78 portion of trap 60 might still not escape from trap 60. This is because, in response to the tipping, the bubble may move either into or out of the plane of the page defined by FIG. 7 (due to the curvature of barrier 62 when viewed from either above or below (e.g. FIG. 3). This movement may be sufficient to allow the bubble to completely stay out of transmission path 54 at all times while it migrates toward the portion of trap 60 adjacent left side 76 (of FIG. 7). An example of this potential movement is illustrated in FIG. 3 by arrows 86 and 88. Depending upon the degree and orientation of the tilting, as well as the initial position of the bubble, it may migrate in either direction 86 or 88 such that it circumnavigates transmission path 54 on its journey toward the opposite side of module 28.

When module 28 is rotated in a clockwise direction (FIG. 7), any bubbles that are initially positioned in trap 60 adjacent left side 76 will remain in the left portion of trap 60. This is true even if module 28 is rotated beyond ninety degrees. Indeed, if module 28 is rotated 180 degrees clockwise (with respect to FIG. 7), any bubbles that are initially positioned in trap 60 adjacent left side 76 will migrate along outer wall 74 until they end up trapped adjacent second barrier 64, as will be discussed in more detail below. However, before turning to a further discussion of second barrier 64, it should be noted that the same type of bubble trapping will occur if the module 28 of FIG. 7 is rotated counterclockwise. That is, the counterclockwise rotation will tend to urge bubbles toward a portion of trap 60 that is near right side 78. If bubbles are initially positioned near left side 76, they may get caught by barrier 62 and prevented from migrating through transmission path 54 unless such counterclockwise rotation exceeds ninety degrees. Even if it does exceed ninety degrees, some bubbles initially on left side 76 of trap 60 may still completely avoid traveling through transmission path 54 by moving into or out of the plane defined by the page of FIG. 7, and thus staying in bubble trap 60 during their complete journey over toward right side 78. Still further, those bubbles initially positioned in trap 60 near right side 78 will remain in trap 60 toward right side 78.

To the extent the counterclockwise rotation of module 28 (as shown in FIG. 7) continues past ninety degrees, the bubbles in trap 60 on the right side 78 of module 28 will continue to migrate along outer wall 74. If such counterclockwise rotation continues for 180 degrees, the bubbles will eventually end up adjacent lower or second barrier 64. Second barrier 64, as will be discussed more below, operates in a similar fashion to first barrier 62, except that it performs its functions when module 28 is tipped upside down (i.e. when wand 22 is oriented such that module 28 is positioned above transducer 26, examples of such an orientation are shown in FIGS. 2 and 4-6).

It will be understood by those skilled in the art that the precise shape of side wall 72 and outer wall 74 may be changed from that illustrated. For example, the shape of side wall 72 could be changed so that it was concavely shaped (as viewed in FIG. 7), and thus it would be able to capture bubbles therein even when module 28 was tipped more than ninety degrees. Further, side wall 72 could have its shape changed so that it did not include any flat portions when viewed from above or below (e.g. FIG. 3). By replacing the flat sections with curved sections, any tipping of the wand 22 would cause bubbles that were abutting side wall 72 to experience a force that tended to urge them to circumnavigate transmission path 54 in either a clockwise or counterclockwise direction (e.g. in the directions of arrows 86 or 88 in FIG. 3). This could help prevent any bubbles from crossing transmission path 54. Still other variations are possible.

It will further be understood by those skilled in the art that the shape of exterior edge 68 of driver portion 46 could be changed from the planar shape illustrated in FIGS. 4-7 to a non-planar shape. Still further, the shape of flat surface 70 could be changed so that it was not horizontal (in the orientation of FIG. 7), but was instead angled upwardly such that its edge closest to side wall 72 was higher than its edge closest to driving member 46 (as viewed in FIG. 7). Either of these variations could facilitate the migration of bubbles toward trap 60 whenever module 28 was positioned in the vertical orientation of FIG. 1 or 7. In these perfectly vertical orientations, any bubbles in the transmission path that migrated up to driver 46 or flat surface 70 might tend to stay within transmission path 54 until module 28 was tipped. However, by angling flat surface 70 and/or changing exterior edge 68 of driver 46 to be non-planar, the bubbles could be directed outwardly toward trap 60.

Barrier 64 will now be described with reference to FIGS. 2 and 4-6. As noted, barrier 64 operates in substantially the same manner as barrier 62. The primary difference is that barrier 64 prevents bubbles from escaping trap 60 when module 28 is delivering ultrasonic energy upwardly (e.g. FIGS. 2 and 4-6), while barrier 62 prevents bubbles from escaping trap 60 when module 28 is delivering ultrasonic energy downwardly (e.g. FIGS. 1 and 7). Just as barrier 62 includes a side wall 72 that obstructs nearby bubbles from migrating into transmission path 54, barrier 64 likewise includes a side wall 80 that functions in the same manner. Thus, when module is tipped in a clockwise or counterclockwise direction (with respect to FIG. 2 or 4-6), side wall 80 of barrier 64 will prevent nearby bubbles from entering transmission path 54 provided such tipping does not exceed ninety degrees from vertical (as defined by gravity). Tipping greater than ninety degrees may allow a bubble to escape into transmission path 54, however, this will depend upon whether the bubble is positioned adjacent a curved section of barrier 64 or a flat section of barrier 64. If positioned along a curved section, the curvature may urge the bubble to travel around, rather than through, transmission path 54. In any event, bubbles traveling through transmission path 54 will likely re-enter trap 60 on the opposite side. Thus, bubbles in trap 60 will be substantially prevented from escaping therefrom for virtually all orientations of wand 22, and even in those orientations where a bubble may cross transmission path 54, the traversal of path 54 will like only be temporary until the bubble re-enters trap 60 on an opposite side.

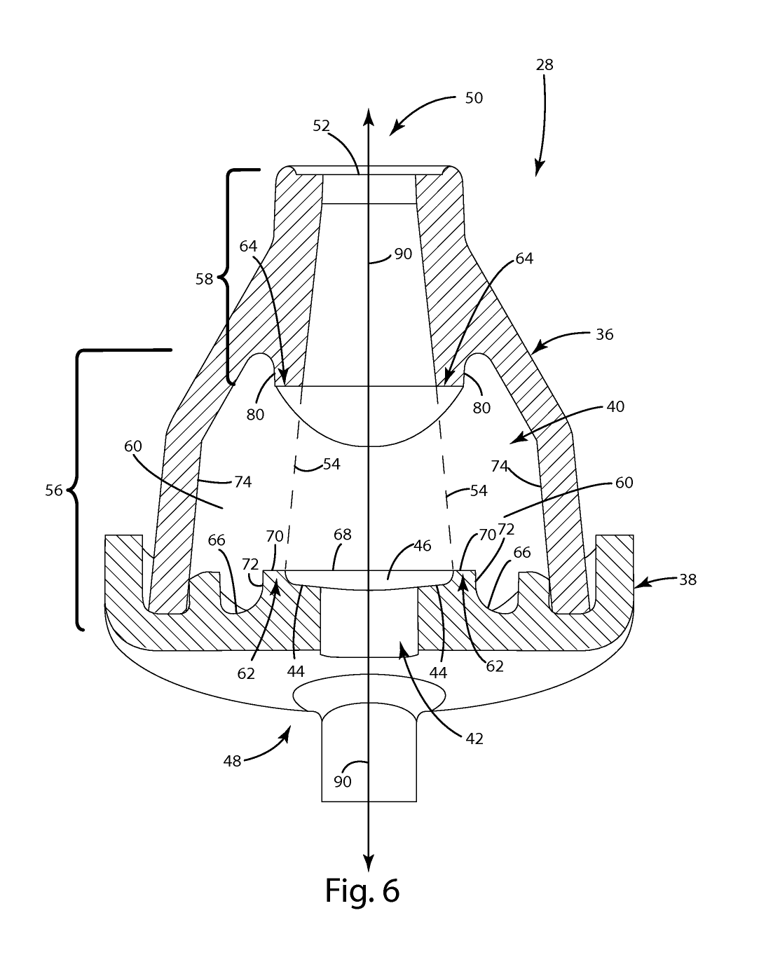

It will be understood that the examples of tipping described herein are not the only types of tipping in which bubbles are retained within trap 60. That is, module 28 may be tipped in other orientations beyond the purely orthogonal directions shown in FIGS. 4-7. For example, module 28 may be tipped a certain amount such that it rotates clockwise in FIG. 4a certain amount, as well as also simultaneously being tipped a certain amount such that it also rotates clockwise in FIG. 6. In such situations where there is a non-zero amount of tipping in two orthogonal directions, barriers 62 and 64 will serve to prevent bubbles from migrating into transmission path in the same manners described above. Indeed, module 28 may have its vertical axis 90 (FIG. 4 or 6) tipped, and then have the component of its vertical axis 90 that is projected into the horizontal plane rotated three-hundred and sixty degrees around a gravitationally vertical axis without having any bubbles escape from traps 60.

As shown more clearly in FIGS. 4, 5, and 7, module 28 may include a plurality of fastener apertures 82 that are adapted to receive fasteners for securing module 28 to transducer 26. Fastener apertures 82 may be internally threaded so as to threadingly mate with threaded fasteners, or they may be configured in different manners. The fasteners inserted into apertures 82 are sufficiently long so as to extend into corresponding apertures (not shown) defined in transducer 26. In this manner, the fasteners can secure module 28 to transducer 26. The securement of module 28 to transducer 26 may be either permanent or temporary. That is, in some embodiments, the fasteners may allow module 28 to be removed from transducer 26 so that the module may be discarded, if no longer useful, or repaired separately from transducer 26. In other embodiments, module 28 may be permanently secured to transducer 26 such that any non-repairable malfunction of either module 28 or transducer 26 will require the entire wand 22 to be discarded.

Cover 38 and base 36 of module 28 may be made from any suitable material. Examples of such suitable materials include polycarbonate, polyetherimide or polypropylene, although it will be understood by those skilled in the art that these examples are not limiting, and that other materials may be used. Cover 38 may be secured to base 36 in any suitable manner. This may involve ultrasonic welding, RF welding, the use of adhesives, or any other techniques that are suitable for creating a fluid-impermeable seal between base 36 and cover 38. In some embodiments, it may be suitable to replace base 36 and cover 38 with a one-piece unit that defines fluid chamber 40 and that includes a sealable aperture for injecting the interior with the ultrasonic fluid. Still other variations are possible.

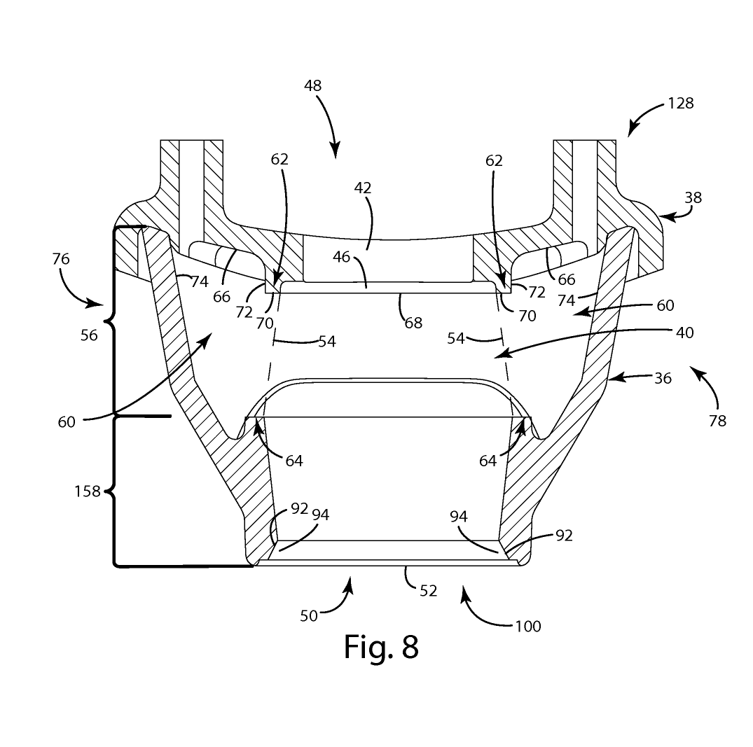

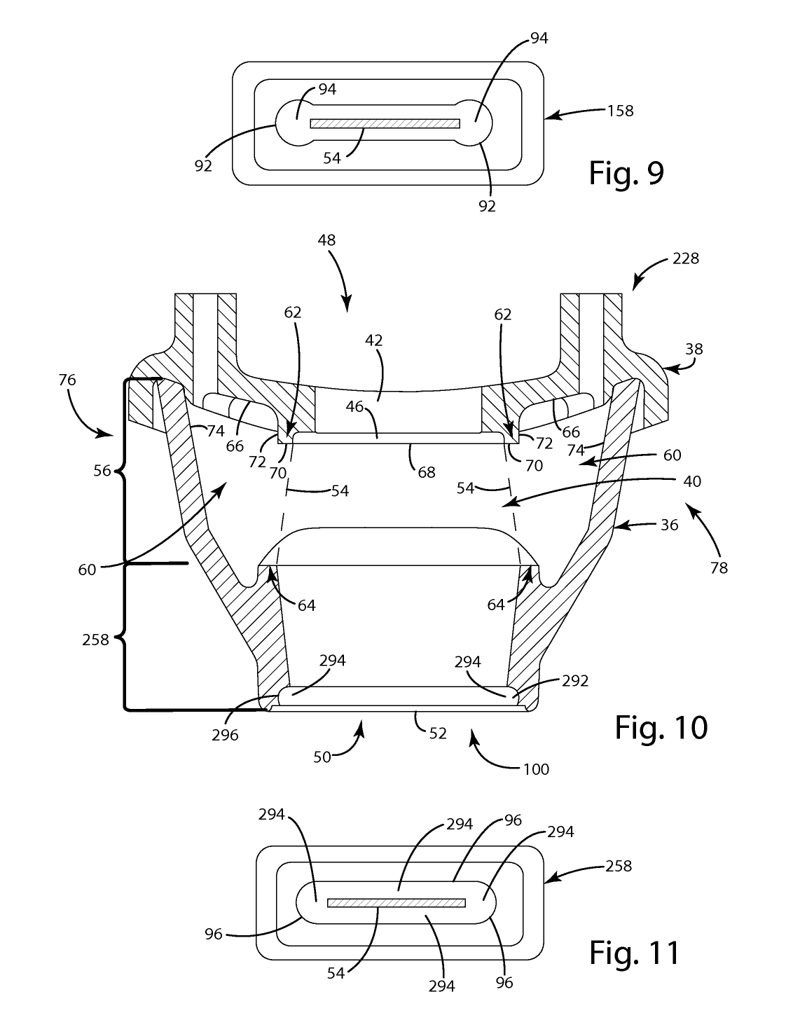

FIGS. 8-9 illustrate a modified ultrasonic module 128 that may be used with the ultrasonic energy delivery system 20 described above, as well as with other ultrasonic energy delivery systems. Modified ultrasonic module 128 includes a substantial number of components that are the same as those found in module 28, and those common components are labeled in FIGS. 8-9 with the same reference numbers used to describe module 28. These common components function in the same manner as has been described above and may be made of the same materials as has been described above with respect to module 28. Accordingly, repeated description of these common components is not necessary. In addition to these common components, modified ultrasonic module 128 also includes several new components that have been labeled with new numbers.

Modified ultrasonic module 128 differs from module 28 in that it includes a modified second section 158 having a delivery end bubble trap 94 positioned adjacent contact membrane 52. The delivery end bubble trap 94 is designed to help remove bubbles from the ultrasonic transmission path 54 that form in, or migrate to, a close proximity of contact membrane 52. In the embodiment shown in FIGS. 8 and 9, delivery end bubble trap 94 is defined by angled sidewalls 92. More specifically, there are two angled sidewalls 92 defined in module 128: a first one of which is positioned adjacent contact membrane 52 along left side 76 and a second one of which is positioned adjacent contact membrane 52 along right side 78. Angled sidewalls 92 provide space for bubbles to migrate to that is not contained within transmission path 54.

The space provided for bubbles by delivery end bubble trap 94 is better shown in FIG. 9, which is an end view of the module 128 of FIG. 8 viewed from the direction of arrow 100 in FIG. 8. Angled sidewalls 92 are shown at right and left sides 78 and 76, respectively, and these create a relatively large gap between transmission path 54 and the angled sidewalls 92. This gap provides space for bubbles to reside which does not interfere with the transmission of ultrasonic energy to membrane 52. Although bubble traps 94 do not include any lips, ledges, or barrier walls that prevent bubbles from migrating out of bubble traps 94 and into transmission path 54, the normal usage of ultrasonic energy delivery system 20 will normally not take place in a perfectly vertical and stationary orientation, such as that shown in FIG. 8. Instead, the module 128 will likely be tipped and moved to non-vertical orientations. When in such non-vertical orientations, any bubbles in transmission path 54 will tend to move to the side (76 or 78) of module 128 that is at a higher elevation. Consequently, such bubbles will tend to move toward the higher of the two bubble traps 94 (when module 128 is oriented generally upside down from the orientation shown in FIG. 8). When module 128 is oriented generally as shown in FIG. 8, or tipped somewhat, any bubbles in traps 94 will tend to slide upward along sidewall 92 and into trap 60.

FIGS. 10-11 illustrate a second modified ultrasonic module 228 that may be used with the ultrasonic energy delivery system 20 described above, as well as with other ultrasonic energy delivery systems. Modified ultrasonic module 228 includes a substantial number of components that are the same as those found in modules 28 and 128, and those common components are labeled in FIGS. 10-11 with the same reference numbers used to describe module 28 and/or module 128. These common components function in the same manner as has been described above and may be made of the same materials as has been described above with respect to module 28 and/or module 128. Accordingly, repeated description of these common components is not necessary.

Ultrasonic module 228 includes a modified second section 258 having a delivery end bubble trap 294 positioned adjacent contact membrane 52. Delivery end bubble trap 294 differs from bubble trap 94 of module 128 in two primary ways. First, delivery end bubble trap 294 includes a curved wall 296 instead of an angled and generally straight sidewall 92. Second, delivery end bubble trap 294 extends circumferentially around the entire transmission path 54, as can best be seen in FIG. 11. That is, instead of residing primarily only at right and left sides 78 and 76, as bubble trap 94 does, bubble trap 294 extends between the right and left side 78 and 76, as well as residing at right and left sides 78 and 76. Tipping ultrasonic module 228 either forwardly or rearwardly out of the plane of the page of FIG. 10 will cause any bubbles positioned nearby to move forwardly or rearwardly out of interference with transmission path 54.

It will be understood by those skilled in the art that bubble traps 94 and/or 294 can be used in still other embodiments where there are no additional bubble traps. That is, in some additional embodiments, module 128 or 228 can be modified so as to exclude bubble trap 60 and only rely on bubble traps 94 and/or 294. It will further be understood by those skilled in the art that the angled walls 92 of module 128 could be replaced with curved walls, such as curved walls 296 of module 228, or other types of curved walls. Similarly, the curved walls 296 of module 228 could be replaced with angled walls, such as angled walls 92 of module 128, or with other types of straight or angled walls. Finally, it will be understood by those skilled in the art that the shape of bubble traps 92 and/or 294 can be varied in other manners from those shown in FIGS. 8-11.

Various other alterations and changes can be made to the foregoing embodiments without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Still further, any references to “first” and “second,” or other numerical designations, should not be construed as excluding additional elements that may be added beyond those specifically identified by number.

What is claimed is:

1. An ultrasonic module for transmitting ultrasonic energy from a transducer to a target, said module comprising:
a first end for receiving ultrasonic energy from an ultrasonic transducer;
a second end for delivering ultrasonic energy to a target;
a fluid chamber positioned between said first and second ends and adapted to contain an ultrasonic fluid; and
a bubble trap adapted to hold bubbles formed in said ultrasonic fluid in a location outside of an ultrasonic energy transmission path defined between said first and second ends.

2. The module of claim 1 further including:
a seat defined at said first end for positioning an ultrasonically vibrating member; and
a barrier surrounding said seat that obstructs bubbles in said bubble trap from escaping out of the bubble trap and into said transmission path.

3. The module of claim 2 wherein said barrier prevent bubbles in said bubble trap from escaping out of the bubble trap and into said transmission path when said module is tipped from a vertical orientation to a tipped orientation, said vertical orientation defined by said first and second ends being vertically aligned, and said tipped orientation being defined by said first and second end not being vertically aligned.

4. The module of claim 3 wherein said tipped orientation includes angular deviations as great as ninety degrees from said vertical orientation.

5. The module of claim 1 wherein said bubble trap is adapted to capture bubbles positioned in the transmission path whenever said module is tipped such that first and second ends are not vertically aligned.

6. The module of claim 1 wherein said bubble trap is positioned closer to said first end than to said second end.

7. The module of claim 1 wherein fluid chamber includes a first section and a second section, said first section including a first inner dimension and said second section including a second inner dimension, said first inner dimension being larger than said second inner dimension.

8. The module of claim 7 wherein said bubble trap is defined in said first section.

9. The module of claim 8 further including a barrier defined adjacent a junction of said first and second sections, said barrier adapted to obstruct bubbles from escaping out of said bubble trap and into said transmission path.

10. The module of claim 1 further including:
a first barrier defined near said first end and adapted to obstruct bubbles in said bubble trap from escaping out of the bubble trap and into said transmission path; and
a second barrier defined between said first barrier and said second end, said second barrier also adapted to obstruct bubbles in said bubble trap from escaping out of the bubble trap and into said transmission path.

11. The module of claim 10 wherein said first barrier is adapted to obstruct bubbles when said first end is positioned higher than said second end, and said second barrier is adapted to obstruct bubbles when said second end is positioned higher than said first end.

12. The module of claim 1 wherein said target is human tissue and said ultrasonic energy is focused to generate heat within the human tissue.

13. The module of claim 12 wherein said second end includes a contact membrane adapted to contact human skin when delivering ultrasonic energy to the human tissue.

14. The module of claim 13 further including a releasable fastening system for releasably securing said module to an ultrasonic transducer such that said module may be discarded without discarding the ultrasonic transducer.

15. The module of claim 1 further including a plurality of apertures adapted to receive a plurality of fasteners having a length sufficient to extend into a plurality of corresponding apertures defined in the ultrasonic transducer such that, when said fasteners are inserted into said apertures and said corresponding apertures, said module is secured to the ultrasonic transducer.

16. The module of claim 1 wherein said ultrasonic fluid is an acoustic gel.

17. The module of claim 1 wherein said fluid chamber and said ultrasonic fluid are further adapted to focus the ultrasonic energy as the ultrasonic energy passes from the first end to the second end.

18. The module of claim 1 wherein said bubble trap includes a first bubble trap positioned adjacent the first end and a second bubble trap positioned adjacent the second end.

19. The module of claim 18 wherein said second bubble trap extends circumferentially around the transmission path.

20. A hand-held ultrasonic wand for delivering heat energy to human tissue using ultrasonic energy, said wand comprising:
an ultrasonic transducer adapted to convert electrical energy into ultrasonic energy;
a fluid chamber coupled to said ultrasonic transducer, said fluid chamber adapted to contain an ultrasonic fluid through which said ultrasonic energy is transmitted; and
a bubble trap defined within said fluid chamber, said bubble trap adapted to hold bubbles formed in said ultrasonic fluid in a location outside of an ultrasonic energy transmission path defined between said ultrasonic transducer and the human tissue.

21. The wand of claim 20 further including:
a first barrier defined in said fluid chamber near said ultrasonic transducer, said first barrier adapted to obstruct bubbles in said bubble trap from escaping out of the bubble trap and into said transmission path; and
a second barrier defined in said fluid chamber and spaced away from said second barrier, said second barrier also adapted to obstruct bubbles in said bubble trap from escaping out of the bubble trap and into said transmission path.

22. The wand of claim 21 wherein said first barrier is adapted to obstruct bubbles when said wand is positioned above the human tissue, and said second barrier is adapted to obstruct bubbles when said wand is positioned below the human tissue.

23. The wand of 21 further including a releasable fastening system for releasably securing said fluid chamber to said ultrasonic transducer such that said fluid chamber may be discarded without discarding the ultrasonic transducer.

24. The wand of claim 21 wherein said ultrasonic fluid is an acoustic gel.

25. The wand of claim 21 further including:
a seat defined in said fluid chamber for receiving a driver of the ultrasonic transducer; and
a contact membrane adapted to contact human skin when delivering ultrasonic energy to the human tissue.

26. The wand of claim 25 wherein said bubble trap is adapted to hold bubbles therein when said wand is tilted up to at least as much as ninety degrees from a vertical orientation, said vertical orientation being defined by vertical alignment of the seat with the contact membrane.

27. The wand of claim 26 wherein said bubble trap is adapted to hold bubbles therein when said wand is rotated three-hundred and sixty degrees around a vertical axis while being tilted up to at least as much as ninety degrees from the vertical axis.

28. The wand of claim 25 wherein said bubble trap is adapted to capture bubbles positioned in the transmission path whenever the wand is tipped such that the seat and the contact membrane are not vertically aligned.

29. The wand of claim 20 wherein said fluid chamber includes a first section and a second section, said first section positioned adjacent the ultrasonic transducer, and said second section positioned away from the ultrasonic transducer, said bubble trap being defined in said first section.

30. The wand of claim 29 wherein said first section includes a first inner dimension and said second section includes a second inner dimension, said first inner dimension being larger than said second inner dimension.

31. The wand of claim 20 wherein said ultrasonic transducer includes a driver portion having a non-planar surface in contact with the ultrasonic fluid, said non-planar surface configured to urge the bubbles toward the bubble trap whenever the bubbles push against the non-planar surface of the driver portion.

32. The wand of claim 20 wherein said fluid chamber and said ultrasonic fluid are further adapted to focus the ultrasonic energy as the ultrasonic energy passes from the transducer toward the human tissue.

33. The wand of claim 25 further including a second bubble trap spaced away from said bubble trap.

34. The wand of claim 33 wherein said bubble trap is positioned adjacent said seat and said second bubble trap is positioned adjacent said contact membrane.

35. A method of delivering ultrasonic energy to a target in order to create heat at the target, said method including:
generating ultrasonic vibrations in a driver portion of an ultrasonic transducer;
transmitting the ultrasonic vibrations of the driver portion through a transmission path of an ultrasonic fluid that is in contact with said driver portion; and
capturing bubbles in a location outside of the transmission path such that the bubbles do not interfere with the transmission of the ultrasonic vibrations through the fluid toward the target.

36. The method of claim 35 further including retaining bubbles in said location outside of the transmission path when the ultrasonic transducer is tilted as much as at least ninety degrees from a vertical orientation, said vertical orientation being defined with respect to gravity.

37. The method of claim 35 further including storing the fluid in a fluid chamber that may be released from the transducer to allow a different fluid chamber to be attached to the transducer.

38. The method of claim 35 further including storing the fluid in a fluid chamber that is permanently affixed to the transducer.

39. The method of claim 35 further including:
retaining bubbles in said location outside of the transmission path when the ultrasonic transducer is tilted as much as at least ninety degrees from a first vertical orientation, said first vertical orientation being defined with respect to gravity and with said transducer positioned above said fluid; and
retaining bubbles in said location outside of the transmission path when the ultrasonic transducer is tilted as much as at least ninety degrees from a second vertical orientation, said second vertical orientation being defined with respect to gravity and with said fluid being positioned above said transducer.
